(19) United States
(12) Patent Application Publication
(10) Pub. No.: US 2020/0268772 A1
(43) Pub. Date: Aug. 27, 2020

(54) HIV MATURATION INHIBITOR FORMULATIONS

(71) Applicant: VIIV HEALTHCARE UK (NO.4) LIMITED, Brentford, Middlesex (GB)

(72) Inventors: Albert J. DELMONTE, Wallingford, CT (US); Ira B. DICKER, Wallingford, CT (US); Carey Kang-Lun HWANG, Wallingford, CT (US); Samit Ramesh JOSHI, Wallingford, CT (US); Max LATAILLADE, Wallingford, CT (US)

(21) Appl. No.: 15/776,461

(22) PCT Filed: Nov. 18, 2016
(86) PCT No.: PCT/IB2016/056956

§ 371 Date: May 16, 2018

Related U.S. Application Data
(60) Provisional application No. 62/257,871, filed on Nov. 20, 2015.
(60) Provisional application No. 62/376,516, filed on Aug. 18, 2016.

(51) Int. Cl. A61K 31/58; A61K 31/4418; A61K 31/427

(52) U.S. Cl. CPC A61K 31/58; A61K 31/427; A61K 31/4418

(57) ABSTRACT

Co-formulations of HIV maturation inhibitor compound with one or two other HIV compounds, and methods of treatment, are set forth.

### FIELD OF THE INVENTION

The invention is directed to formulations useful against HIV containing two and three drug combinations of antiretroviral compounds. In particular, the invention is directed to combinations of an HIV maturation inhibitor compound, and one or two other antiretroviral compounds, including dolutegravir and atazanavir. The invention is also directed to methods of administering these formulations to patients in need of treatment.

### BACKGROUND OF THE INVENTION

HIV-1 (human immunodeficiency virus-1) infection remains a major medical problem, with tens of millions of people still infected worldwide at the end as of 2013. The number of cases of HIV and AIDS (acquired immunodeficiency syndrome) has risen rapidly. In 2005, approximately 5.0 million new infections were reported, and 3.1 million people died from AIDS. Currently available drugs for the treatment of HIV include nucleoside reverse transcriptase (RT) inhibitors or approved single pill combinations: zidovudine (or AZT or RETROVIR®), didanosine (or VIDEX®), stavudine (or ZERIT®), lamivudine (or 3TC or EPIVIR®), zalcitabine (or DDC or HIVID®), abacavir succinate (or ZIAGEN®), Tenofovir disoproxil fumarate salt (or VIREAD®), emtricitabine (or FTC—EMTRIVA®), COMBIVIR® (contains -3TC plus AZT), TRIZIVIR® (contains abacavir, lamivudine, and zidovudine), EPZICOM® (contains abacavir and lamivudine), TRUVADA® (contains VIREAD® and EMTRIVA®); non-nucleoside reverse transcriptase inhibitors: nevirapine (or VIRAMUNE®), delavirdine (or RESCRIPTOR®) and efavirenz (or SUSTIVA®), ATRIPLA® (TRUVADA®+SUSTIVA®), and etravirine, and peptidomimetic protease inhibitors or approved formulations: saquinavir, indinavir, ritonavir, nelfinavir, amprenavir, lopinavir, KALETRA® (lopinavir and Ritonavir), darunavir, atazanavir (REYATAZ®) and tipranavir (APTIVUS®) and cobicistat, and integrase inhibitors such as raltegravir (ISENTRESS®) and dolutegravir (not yet approved), and entry inhibitors such as enfuvirtide (T-20) (FUZEON®) and maraviroc (SELZENTRY®).

Each of these drugs can only transiently restrain viral replication if used alone. However, when used in combination, these drugs have a profound effect on viremia and disease progression. In fact, significant reductions in death rates among AIDS patients have been recently documented as a consequence of the widespread application of combination therapy. However, despite these impressive results, 30 to 50% of patients may ultimately fail combination drug therapies. Insufficient drug potency, non-compliance, restricted tissue penetration and drug-specific limitations within certain cell types (e.g. most nucleoside analogs cannot be phosphorylated in resting cells) may account for the incomplete suppression of sensitive viruses. Furthermore, the high replication rate and rapid turnover of HIV-1 combined with the frequent incorporation of mutations, leads to the appearance of drug-resistant variants and treatment failures when sub-optimal drug concentrations are present. Therefore, novel anti-HIV agents exhibiting distinct resistance patterns, and favorable pharmacokinetic as well as safety profiles are needed to provide more treatment options.

Another emerging class of compounds for the treatment of HIV are called HIV maturation inhibitors. Maturation is the last step in HIV replication or the HIV life cycle, in which HIV becomes infectious as a consequence of several HIV protease-mediated cleavage events in the gag protein that ultimately results in release of the capsid (CA) protein. Maturation inhibitors bind to the Gag polyprotein of budding virus, blocking a key protease cleavage event and thereby blocking maturation. Thus, maturation inhibitors block the last protease cleavage event between Gag protein segments designated as capsid (CA) protein p24 (p24) and spacer peptide 1 (SP1), resulting in the release of immature noninfectious virus particles, preventing subsequent cycles of HIV infection.

Certain derivatives of betulinic acid have now been shown to exhibit potent anti-HIV activity as HIV maturation inhibitors. In particular, reference is made herein to the pending patent application by Bristol-Myers Squibb entitled “C-17 AND C-3 MODIFIED TRITERPENOIDS WITH HIV MATURATION INHIBITORY ACTIVITY” with Ser. No. 13/359,727, filed on Jan. 27, 2012 (now U.S. Pat. No. 8,846,647), incorporated herein by reference.

Also important as agents against HIV are the aforementioned nucleoside reverse transcriptase inhibitors, or NRTIs. Also of note is the compound with the generic name festinavir, which is set forth and claimed in U.S. Pat. No. 7,589,078, also incorporated herein. The protease inhibitors, such as atazanavir, are also highly efficacious against HIV. In addition, the integrase inhibitors, particularly dolutegravir, have also emerged as potent agents with documented activity against HIV.

What is now needed in the art are new formulations which are useful in the treatment against HIV, and include one or more HIV maturation inhibitors, as well as one or two other potent antiretroviral drugs. These new 2 and 3-drug formulations should be convenient and easy to administer, and provide optimal dosing of important HIV medications.

### SUMMARY OF THE INVENTION

In a first embodiment, the invention is directed to a three drug formulation of antiretroviral drugs useful against HIV, comprising a maturation inhibitor compound, an integrase inhibitor compound and a protease inhibitor compound.

In a second embodiment, the invention is directed to a three drug formulation of antiretroviral drugs useful against HIV, comprising the maturation inhibitor compound having the structural formula

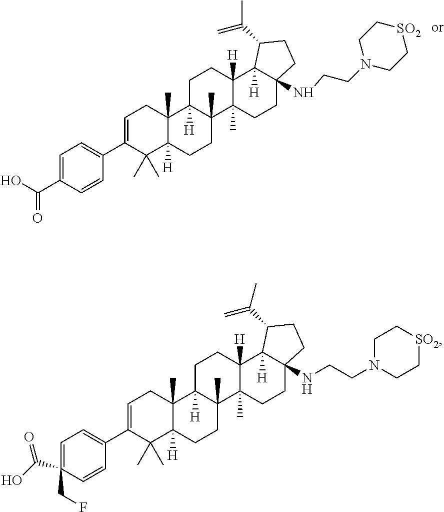

as well as dolutegravir and atazanavir, preferably unboosted.

In a third embodiment, the invention is directed to a three drug formulation of antiretroviral drugs useful against HIV, comprising a maturation inhibitor, an integrase inhibitor compound, and an NRTI compound.

In a fourth embodiment, the invention is directed to a three drug formulation of antiretroviral drugs useful against HIV, comprising the HIV maturation inhibitor compounds:

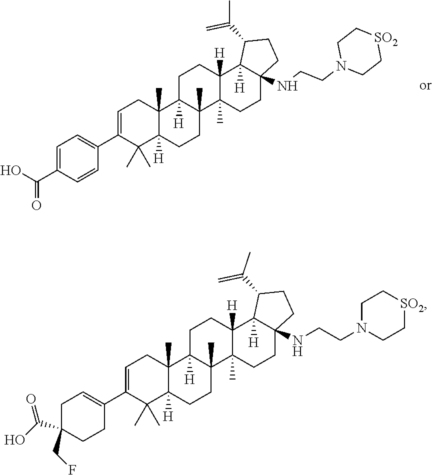

as well as atazanavir and doravirine (or another suitable non-nucleoside reverse transcriptase inhibitor).

In a fifth embodiment, the invention is directed to a three drug formulation of antiretroviral drugs useful against HIV, comprising a maturation inhibitor compound, a protease inhibitor compound, and an NRTI compound or a NNRTI compound.

In a sixth embodiment, the invention is directed to a three drug formulation of antiretroviral drugs useful against HIV, comprising the HIV maturation inhibitor compound; as well as atazanavir and tenofovir.

The invention is also directed to two drug formulations useful against HIV comprising a maturation inhibitor compound, and one other agent such as an integrase inhibitor or a protease inhibitor. For example, one formulation will comprise the HIV maturation inhibitor compound

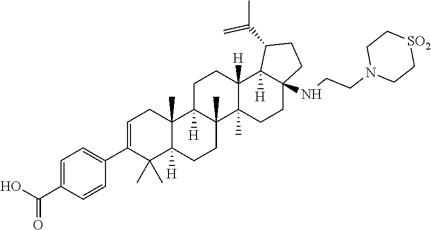

or the maturation inhibitor compound

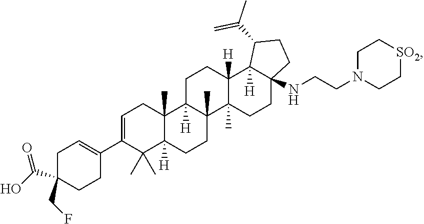

as well as a protease inhibitor such as atazanavir. The protease inhibitor may be boosted or unboosted with another compound such as ritonavir, but is preferably unboosted. Other example of a suitable two drug formulation will include the HIV maturation compound above in combination with the integrase inhibitor dolutegravir.

By way of non-limiting example, a two drug formulation could include about 40 mg. of the HIV maturation inhibitor compound, along with 400 mg. of atazanavir. Another two drug formulation could include about 80 mg. of the HIV maturation inhibitor compound, along with 400 mg. of atazanavir. Another suitable formulation could include about 40 mg. of the HIV maturation inhibitor, along with 300 mg. of atazanavir (which is boosted with 100 mg. of ritonavir).

Other two and three drug formulations could include a maturation inhibitor (as set forth above), further in combination with one or more other HIV compounds in development, including allosteric integrase inhibitors (ALLINIs) and HIV capsid compounds. These could also be combined with an integrase inhibitor, such as dolutegravir (DTG). Thus, some possible combinations are represented in the following table:

| FDC (Two drug) | STR (Three drug) |
|---|---|
| HIV Maturation + ALLINI | HIV Maturation + DTG + ALLINI |
| HIV Maturation + Capsid | HIV Maturation + DTG + Capsid |
| ALLINI + Capsid | HIV Maturation + ALLINI + Capsid |
| DTG + ALLINI | DTG + ALLINI + Capsid |
| DTG + Capsid | |

The invention is further directed to methods of treatment using the combination drug formulations herein set forth.

These and other objects of the invention will become apparent in the ensuing description and the appended claims.

### DETAILED DESCRIPTION OF THE EMBODIMENTS

The formulations of the present invention, according to all the various embodiments described above, may be administered orally, parenterally (including subcutaneous injections, intravenous, intramuscular, intrasternal injection or infusion techniques), by inhalation spray, or rectally, and by other means, in dosage unit formulations containing non-toxic pharmaceutically acceptable carriers, excipients and diluents available to the skilled artisan. One or more adjuvants may also be included.

The pharmaceutical formulations of the invention may be in the form of orally administrable suspensions or tablets; as well as nasal sprays, sterile injectable preparations, for example, as sterile injectable aqueous or oleaginous suspensions or suppositories. Pharmaceutically acceptable carriers, excipients or diluents may be utilized in the pharmaceutical compositions, and are those utilized in the art of pharmaceutical preparations.

When administered orally as a suspension, these compositions are prepared according to techniques typically known in the art of pharmaceutical formulation and may contain microcrystalline cellulose for imparting bulk, alginic acid or sodium alginate as a suspending agent, methylcellulose as a viscosity enhancer, and sweeteners/flavoring agents known in the art.

As tablets, these formulations may contain, by way of non-limiting examples, microcrystalline cellulose, hydroxymethyl cellulose, hydroxyethyl cellulose, hydroxypropyl cellulose (HPC), hydroxypropyl methyl cellulose (HPMC), and/or other available excipient polymers, as well as dicalcium phosphate, starch, magnesium stearate and lactose and/or other excipients, binders, extenders, disintegrants, diluents, and lubricants available to the artisan. In certain embodiments, micronized crystalline HCl salt may also be suitable.

The injectable solutions or suspensions may be formulated, using suitable non-toxic, parenterally acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

Each of the compounds herein set forth as part of the formulations of the invention, can be administered orally to humans in a dosage range of about 1 to 100 mg/kg body weight one or more times daily, usually over an extended period, such as days, weeks, months, or even years. One preferred dosage range is about 1 to 10 mg/kg body weight orally per dose. Another preferred dosage range is about 1 to 20 mg/kg body weight orally per dose. Preferably, the formulations herein can be compounded into once daily, once weekly or even once monthly or longer dosage forms, containing the 2 or 3 drug combinations herein set forth.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy, as well as other possible factors.

Thus, in accordance with the present invention, there is further provided a method of treatment, and a pharmaceutical formulation, for treating viral infections such as HIV infection and AIDS. The treatment involves administering to a patient in need of such treatment one or more of the pharmaceutical formulations herein set forth, which contain an antiviral effective amount of at least two, and preferably three antiretroviral compounds, together with one or more pharmaceutically acceptable carriers, excipients or diluents. As used herein, the term “antiviral effective amount” means the total amount of each active component of the composition and method that is sufficient to show a meaningful patient benefit, i.e., inhibiting, ameliorating, or healing of acute conditions characterized by inhibition of the HIV infection. When applied to an individual active ingredient, administered alone, the term refers to that ingredient alone. When applied to a combination, the term refers to combined amounts of the active ingredients that result in the therapeutic effect, whether administered in combination, serially or simultaneously. The terms “treat, treating, treatment” as used herein and in the claims means preventing, ameliorating or healing HIV and/or diseases associated with HIV infection.

The foregoing description is merely illustrative and should not be understood to limit the scope or underlying principles of the invention in any way. Indeed, various modifications of the invention, in addition to those shown and described herein, will become apparent to those skilled in the art from the following examples and the foregoing description. Such modifications are also intended to fall within the scope of the appended claims.

1-15. (canceled)

16. A two drug formulation of antiretroviral drugs useful against HIV, comprising dolutegravir and a maturation inhibitor selected from the group of:

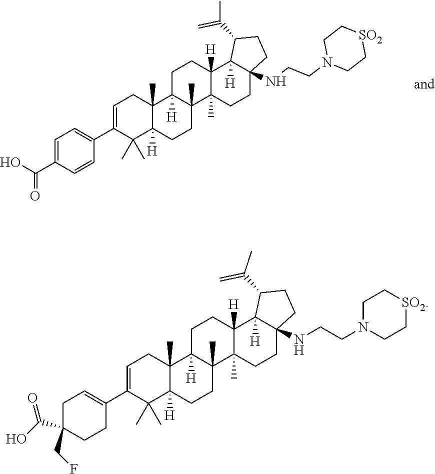

17. A two drug formulation of antiretroviral drugs useful against HIV, comprising atazanavir and a maturation inhibitor selected from the group of:

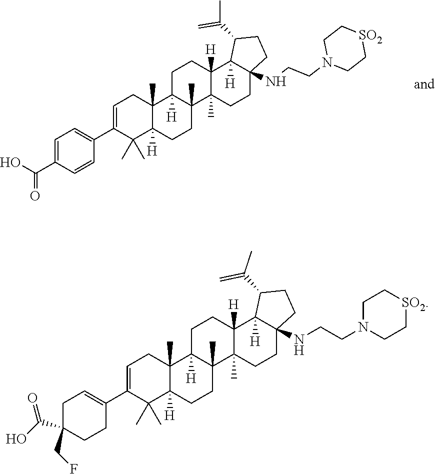

18. The formulation of claim 17, wherein said atazanavir is unboosted.

19. The formulation of claim 17, wherein said atazanavir is boosted with ritonavir.

20-27. (canceled)

28. A method of treating HIV infection, comprising administering to a patient in need the formulation as claimed in claim 16.

29. (canceled)
